(19) United States
(12) Patent Application Publication
(10) Pub. No.: US 2007/0110782 A1
(43) Pub. Date: May 17, 2007

(54) L-HISTIDINE IN OPHTHALMIC SOLUTIONS

(75) Inventor: Francis X. Smith, Salem, NH (US)

Correspondence Address: HISCOCK & BARCLAY, LLP, 2000 HSBC PLAZA, 100 Chestnut Street, ROCHESTER, NY, 14604-2404, US

(73) Assignee: FXS Ventures, LLC

(21) Appl. No.: 11/613,061

(22) Filed: Dec. 19, 2006

Related U.S. Application Data
(63) Continuation-in-part of application No. 10/544,154, filed on Aug. 1, 2005.
(60) Provisional application No. 60/246,689, filed on Nov. 8, 2000.
(60) Provisional application No. 60/246,707, filed on Nov. 8, 2000.
(60) Provisional application No. 60/246,708, filed on Nov. 8, 2000.
(60) Provisional application No. 60/246,709, filed on Nov. 8, 2000.

(51) Int. Cl. A61K 33/40; A61K 31/4172

(52) U.S. Cl. 424/405; 424/616; 514/400

(57) ABSTRACT

The invention relates to an aqueous ophthalmic solution comprising 0.00001 to about 10.0 percent by weight L-histidine, 0.0001 to 3.0 percent by weight hydrogen peroxide, and optionally 0.1 to 500 parts per million of a preservative that provides superior preservative efficacy especially as against fungal microbes. These solutions may be employed in various ways including cleaning contact lenses, rinsing lenses while in the eye, storing lenses and in delivering active pharmaceutical agents to the eye.

### CROSS-REFERENCE TO RELATED APPLICATIONS

This application claims the benefit of U.S. Provisional Patent Application Ser. Nos. 60/246,689, filed Nov. 8, 2000, 60/246,707, filed Nov. 8, 2000, 60/246,708, filed Nov. 8, 2000, and 60/246,709, filed Nov. 8, 2000; PCT Application Serial No. US2001/0046762, filed Nov. 8, 2001, and U.S. patent application Ser. No. 10/544,154, filed Aug. 1, 2005.

### FIELD OF THE INVENTION

The present invention relates to the field of ophthalmic solutions used to treat eyes, store contact lenses, or condition medical devices used in the eye. Such solutions are well known and widely employed with numerous products available commercially. There are several types of solutions within the field depending upon specific use. For instance, there are specific solutions for disinfecting contact lenses, solutions for cleaning contact lenses, solutions for treating the surface of contact lenses, solutions for rinsing lenses, solutions for wetting eyes, etc.

While each of these lenses are formulated specifically for their intended application, each solution is formulated or handled so that it will remain free of sources of infection to they eye. Numerous approaches to this problem have been employed, from methods that call for sterilization of the solution and packaging of the solution in a container that will not allow contamination. Use of specific preservative agents employed in concentrations sufficient to prevent microbial increase have been employed. Oxidative agents have been used as well as methods of irradiation. In the cases where chemical agents have been employed, there has been a tendency to employ one preservative agent in the formulation. It has been found that use of two or more specific agents in combination surprisingly provide greater efficacy in preserving solutions than state of the art single preservative systems and in particular the use of the combination of a cationic polymeric preservative, hydrogen peroxide and L-histidine provide increased preservative efficacy against fungal contamination.

This surprising effect is achievable with the further use of certain, but not all, contact lens solution agents. In particular, certain tonicity agents when employed decrease the preservative efficacy of the invention and should not be employed.

As described in U.S. Pat. No. 4,029,817, hydrophilic plastic materials are used in making soft contact lenses. U.S. Pat. No. 3,503,393 to Seiderman and U.S. Pat. No. 2,976,576 to Wichterle describe processes for producing hydrophilic polymers of polyhydroxyethylmethacrylate in aqueous reaction media having a sparingly cross-linked polymeric hydrogel structure and being elastic, soft, transparent hydrogels. Other soft contact lenses are made of silicone and other suitable materials.

Hydrophilic lenses are particularly useful in opthalmology due to their ability to absorb water and swell to a soft mass of good mechanical strength, and due to their transparency with the ability to retain shape and dimensions when equilibrated in ocular fluid and in storage fluids when removed from the eye.

One problem with soft contact lenses, however, is their sterilization and cleaning. The property of hydrophilic soft lenses which allows them to absorb large amounts of water also allows preservatives which might otherwise be used for cleaning and sterilization to be absorbed and later released onto the eye. The release, furtheremore, may be much slower than the intake, thereby allowing preservatives to build up in the lenses. This can have the harmful result of damaging or staining contact lenses or harming the sensitive tissues of the conjunctivae or cornea.

As stated by R. E. Phares in U.S. Pat. No. 3,689,673, sterilization of hydrophilic soft contact lenses may be carried out by soaking in an aqueous solution containing approximately 0.001-0.01% chlorhexidine for a time sufficient to sterilize the lens.

Various related methods are disclosed in other U.S. patents. U.S. Pat. No. 3,591,329 discloses the use of a cationic resin exchange material impregnated with active metallic silver. U.S. Pat. No. 3,755,561 teaches using an aqueous solution of polyvinyl pyrrolidone, a polyalkylene glycol and thimerosal. U.S. Pat. No. 3,873,696 discloses using a combination of potassium peroxymonosulfate in the presence of sodium chloride. In U.S. Pat. No. 3,876,768 is described the use of a chlorinated trisodium phosphate material which is similar to hypochlorite. U.S. Pat. No. 3,888,782 relates to the using of chlorhexidine and polyvinyl pyrrolidone. The use of an iodoform solution containing iodine, polyvinyl alcohol and boric acid is disclosed in U.S. Pat. No. 3,911,107. U.S. Pat. No. 3,912,450 proposes using a combination of an alcoholic glutaraldehyde solution containing a surfactant in conjunction with an ultrasonic radiation device.

U.S. Pat. No. 3,888,782 more particularly discloses an aqueous, substantially isotonic cleaning and sterilizing solution for plastic hydrophilic soft contact lenses containing, as active ingredients, chlorhexidine and polyvinylpyrrolidone. The solution is said to be non-toxic to the eye of the wearer of soft contact lenses and in the presence of a suitable amount of water soluble polyhydroxyethylmethacrylate to prevent the build-up of opaque deposits on the surfaces of soft contact lenses.

U.S. Pat. No. 4,029,817 discloses that soft contact lenses may be sterilized by contacting soft lenses with a sterile, aqueous, substantially isotonic solution containing as an active ingredient, an effective amount of a specific quaternary ammonium compound.

U.S. Pat. No. 4,758,595 teaches a preserving solution comprising a microbicidally or fungicidally effective amount of a biguanide or water-soluble salt thereof, in combination with a buffer system but does not recognize the need to provide a broad spectrum preservative efficacy.

U.S. Pat. No. 4,361,548 discloses and claims disinfecting and/or preserving solution for contact lenses containing 0.00001 to 0.1 weight percent of a dimethyldiallylammonium chloride homopolymer having a molecular weight from about 10,000 to about 1,000,000, optionally together with up to 0.5 weight percent of ethylenediaminetetraacetic acid or other enhancers and optional buffers and the like, but also does not teach a multiple component preservative.

U.S. Pat. No. 4,354,952 is directed to a disinfecting and/or preserving solution for contact lenses containing 0.0035 to 0.04 weight percent of an amphoteric surfactant in combination with 0.0005 to 0.01 weight percent of chlorhexidine and 0.002 to 0.025 weight percent of a non-ionic surfactant, optionally together with up to 0.5 weight percent of thimerosal or other enhancers and optional buffers and the like. While a multiple preservative system is disclosed, there is no teaching that the system has more than cumulative advantage.

U.S. Pat. No. 5,741,817 broadly teaches the use of amino acids, but is specifically addressed to the use of glycine in combination with specific antimicrobial preservatives, not the specific agents employed in the present invention.

U.S. Pat. No. 6,022,732 teaches that effective hydrogen peroxide based solutions used to disinfect lenses need to be reduced. In particular the patent is directed to Compositions, and methods for using such compositions, which are useful to destroy hydrogen peroxide in a liquid aqueous medium, such as that used to disinfect contact lenses. In one embodiments the composition comprises a hydrogen peroxide destroying component effective when released in a hydrogen peroxide-containing liquid aqueous medium to destroy or cause the destruction of hydrogen peroxide present in the hydrogen peroxide-containing liquid aqueous medium, and a barrier component acting to substantially prevent the release of the hydrogen peroxide destroying component for a period of time after the composition is initially contacted with the hydrogen peroxide-containing liquid aqueous medium, the barrier component comprising a material selected from the group consisting of water soluble cellulose derivatives and mixtures thereof having a molecular weight of at least about 20,000. The composition results in reduced foam formation relative to a similar composition including a barrier component comprising a similar material having a molecular weight of 10,000 when both the composition and the similar composition are exposed to identical hydrogen peroxide-containing liquid aqueous media to destroy or cause the destruction of the hydrogen peroxide therein.

Similarly directed U.S. Pat. No. 5,660,862 teaches a composition useful for disinfecting a contact lens comprising a substantially isotonic, aqueous liquid medium containing hydrogen peroxide in an amount effective to disinfect a contact lens contacted with the aqueous liquid medium, and a hydrogen peroxide reducing agent dissolved in the aqueous liquid medium in an amount effective to enhance the antimicrobial activity of the aqueous liquid medium. Preferably, the composition further includes transition metal ions in an amount effective to further enhance the antimicrobial activity of the aqueous liquid medium and is substantially free of peroxidase

U.S. Pat. No. 5,854,303 teaches a polymeric material incorporating a polyvalent cation chelating agent in an amount effective to inhibit the growth of an ocular pathogen, particularly a protozoan, can be used to produce eye care products such as contact lens cases and containers for containing eye care solutions and contact lenses.

U.S. Pat. No. 4,863,900 teaches that a composition for reducing the transmissability of viral infection from a subject infected therewith which comprises a topically applicable, pharmaceutically acceptable carrier and a viricidally effective amount of a polypeptide of between 24 and 500 aminoacid residues comprising at least 24 residues of L-Histidine. It does not suggest that L-histidine could be used with other bactericidal agents to improve their effect.

U.S. Pat. No. 5,741,817 demonstrates that glycine enhances the activity of antimicrobial preservatives, and could be used in ophthalmic solutions and are useful as substitutes for EDTA, while U.S. Pat. No. 5,494,937 teaches solutions tat contain a combination of glycien with a borate-polyol complex, one or more anionic or nonionic surfactants, and a low molecular weight amino acid (e.g., glycine). This system requires certain anti-bacterial surfactants and no edta. specifically teaches glycine.

U.S. Pat. No. 5,925,317 further shows the use histidine to neutralize iodine in a two step method to avoid lens discloration. The patent teaches that “histidine is not known to have been previously suggested for use in care regimens for contact lenses, although the oxidation reaction of histidine with an excess of iodine is discussed in a paper by Schutte, L., et al, “The Substitution Reaction of Histidine and Some Other Imidazole Derivatives With Iodine,” Tetrahedron, Suppl. 7, pp. 295-306 (1965). One drawback to using an imidazole such as histidine is the formation of an oxidation product that decomposes to a brown degradation product.”

U.S. Pat. No. 6,008,195 returns to the use of polymeric anti-bacterials that have L-histidine as side chain group in the active agent.

### SUMMARY OF THE INVENTION

The invention relates to an aqueous ophthalmic solution comprising 0.00001 to about 10.0 percent by weight histidine, 0.0001 to 3.0 percent by weight hydrogen peroxide, and optionally 0.1 to 500 parts per million of a preservative that provides superior preservative efficacy especially as against fungal microbes. These solutions may be employed in various ways including cleaning contact lenses, rinsing lenses while in the eye, storing lenses and in delivering active pharmaceutical agents to the eye.

The invention may also further comprise a surface-active agent chosen from those known in the art, but in particular might be a hydroxy-ethoxylated castor oil.

The solution can be sued to deliver a pharmaceutical agent to the eye by providing the agent to the solution and then contacting the eye with the resultant solution. Or the solution can be used to clean, treat or store contact lenses by contacting the solution with the contact lens.

One of the objectives of the invention is to provide an acceptable solution that has a greater kill rate than state of the art solutions.

Another object of the invention is to provide an ophthalmic solution which is effective over a broader range of microbial organisms than state of the art solutions.

### DETAILED DESCRIPTION

The invention relates to an aqueous ophthalmic solution comprising 0.00001 to about 10.0 percent by weight L-histidine, 0.0001 to 3.0 percent by weight hydrogen peroxide, and optionally, 0.1 to 500 parts per parts by weight of a preservative that provides superior preservative efficacy, especially as against fungi. These solutions may be employed in various ways including cleaning contact lenses, rinsing lenses while in the eye, storing lenses and in delivering active pharmaceutical agents to the eye. The invention may also further comprise a surface-active agent chosen from those known in the art, but in particular might be a hydroxy-ethoxylated castor oil.

Histidine is a basic amino acid well known in the chemical arts and available from numerous commercial sources. Histidine is known to be used in ophthalmic ointments and the like in very concentrated forms see U.S. Pat. No. 5,811,446.

The cationic polymeric preservatives The cationic polymeric preservative includes polymeric biguanides such as polymeric hexamethylene biguanides (PHMB), and combinations thereof. Such cationic polymeric biguanides, and water-soluble salts thereof, having the following formula: 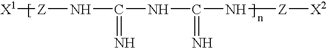
wherein Z is an organic divalent bridging group which may be the same or different throughout the polymer, n is on average at least 3, preferably on average 5 to 20, and X¹ and X² are

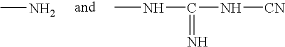

One preferred group of water-soluble polymeric biguanides will have number average molecular weights of at least 1,000 and more preferably will have number average molecular weights from 1,000 to 50,000. Suitable water-soluble salts of the free bases include, but are not limited to hydrochloride, borate, acetate, gluconate, sulfonate, tartrate and citrate salts.

The above-disclosed biguanides and methods of preparation are described in the literature. For example, U.S. Pat. No. 3,428,576 describes the preparation of polymeric biguanides from a diamine and salts thereof and a diamine salt of dicyanimide.

Most preferred are the polymeric hexamethylene biguanides, commercially available, for example, as the hydrochloride salt from Zeneca (Wilmington, Del.) under the trademark Cosmocil™ CQ. Such polymers and water-soluble salts are referred to as polyhexamethylene (PHMB) or polyaminoptopyl biguanide (PAPB). The term polyhexamethylene biguanide, as used herein, is meant to encompass one or 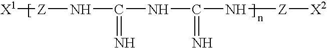
more biguanides have the following formula:
wherein Z, X¹ and X² are as defined above and n is from 1 to 500.

Depending on the manner in which the biguanides are prepared, the predominant compound falling within the above formula may have different X¹ and X² groups or the same groups, with lesser amounts of other compounds within the formula. Such compounds are known and are disclosed in U.S. Pat. No. 4,758,595 and British Patent 1,432,345, which patents are hereby incorporated. Preferably, the water-soluble salts are compounds where n has an average value of 2 to 15, most preferably 3 to 12.

In another embodiment, a polymeric biguanide is used in combination with a bis(biguanide) compound. Polymeric biguanides, in combination with bisbiguanides such as alexidine, are effective in concentrations as low as 0.00001 weight percent (0.1 ppm). It has also been found that the bactericidal activity of the solutions may be enhanced or the spectrum of activity broadened through the use of a combination of such polymeric biguanides with alexidine or similar biguanides.

An optional non-biguanide disinfectant/gennicide can be employed as a solution preservative, but it may also function to potentiate, complement or broaden the spectrum of microbiocidal activity of another germicide. This includes microbiocidally effective amounts of germicides which are compatible with and do not precipitate in the solution, in concentrations ranging from about 0.00001 to about 0.5 weight percent, and more preferably, from about 0.0001 to about 0.1 weight percent. Suitable complementary germicidal agents include, but are not limited to, quaternary ammonium compounds or polymers, thimerosal or other phenylmercuric salts, sorbic acid, alkyl triethanolamines, and mixtures thereof. Representative examples of the quaternary ammonium compounds are compositions comprised of benzalkonium halides or, for example, balanced mixtures of n-alkyl dimethyl benzyl ammonium chlorides. Other examples include polymeric quaternary ammonium salts used in ophthalmic applications such as poly[(dimetlhyliminio)-2-butene-1,4-diyl chloride], [4-tris(2-hydroxyethyl) ammonio]-2-butenyl-w-[tris(2-hydroxyethyl)ammonio]dichloride (chemical registry number 75345-27-6) generally available as polyquaternium I (r) from ONYX Corporation, or those described in U.S. Pat. No. 6,153,568.

Peroxide sources may also be included in the formulations of the present invention and are exemplified by hydrogen peroxide, and such compounds, which provide an effective resultant amount of hydrogen peroxide, such as sodium perborate decahydrate, sodium peroxide, urea peroxide and peracetic acid, an organic peroxy compound.

The pH of the present solutions should be maintained within the range of 5.0 to 8.0, more preferably about 6.0 to 8.0, most preferably about 6.5 to 7.8. Suitable buffers may be added, such as boric acid, sodium borate, potassium citrate, citric acid, sodium bicarbonate, bis-tris propane, TRIS, and various mixed phosphate buffers (including combinations of Na₂HPO₄, NaH₂PO₄ and KH₂PO₄) and mixtures thereof. Borate buffers are preferred, particularly for enhancing the efficacy of PAPB. Generally, buffers will be used in amounts ranging from about 0.05 to 2.5 percent by weight, and preferably, from 0.1 to 1.5 percent.

The solutions of the present invention may further contain other additives including but not limited to buffers, tonicity agents, demulcents, wetting agents, preservatives, sequestering agents (chelating agents), surface active agents, and enzymes.

Ophthalmologically acceptable chelating agents useful in the present invention include amino carboxylic acid compounds or water-soluble salts thereof, including ethylenediaminetetraacetic acid, nitrilotriacetic acid, diethylenetriamine pentaacetic acid, hydroxyethylethylenediaminetriacetic acid, 1,2-diaminocyclohexanetetraacetic acid, ethylene glycol bis (beta-aminoethyl ether) in N, N, N′, N′ tetraacetic acid (EGTA), aminodiacetic acid and hydroxyethylamino diacetic acid. These acids can be used in the form of their water soluble salts, particularly their alkali metal salts. Especially preferred chelating agents are the di-, tn- and tetra-sodium salts of ethylenediaminetetraacetic acid (EDTA), most preferably disodium EDTA (Disodium Edetate).

Other chelating agents such as citrates and polyphosphates can also be used in the present invention. The citrates which can be used in the present invention include citric acid and its mono-, di-, and tri-alkaline metal salts. The polyphosphates which can be used include pyrophosphates, triphosphates, tetraphosphates, trimetaphosphates, tetrametaphosphates, as well as more highly condensed phosphates in the form of the neutral or acidic alkali metal salts such as the sodium and potassium salts as well as the ammonium salt.

The solutions of the invention are compatible with both rigid gas permeable and hydrophilic contact lenses and other ophthalmic devices and instruments during storage, cleaning, wetting, soaking, rinsing and disinfection.

A typical aqueous solution of the present invention may contain additional ingredients which would not affect the basic and novel characteristics of the active ingredients described earlier, such as tonicity agents, surfactants and viscosity inducing agents, which may aid in either the lens cleaning or in providing lubrication to the eye. Suitable tonicity agents include sodium chloride, potassium chloride, glycerol or mixtures thereof The tonicity of the solution is typically adjusted to approximately 240-310 milliosmoles per kilogram solution (mOsm/kg) to render the solution compatible with ocular tissue and with hydrophilic contact lenses. In one embodiment, the solution contains 0.01 to 0.35 weight percent sodium chloride.

The solutions employed in the present invention may also include surfactants such as a polyoxyethylene-polyoxypropylene nonionic surfactant which, for example, can be selected from the group of commercially available surfactants having the name poloxamine or poloxamer, as adopted by The CTFA International Cosmetic Ingredient Dictionary. The poloxamine surfactants consist of a poly(oxypropylene)-poly(oxyethylene) adduct of ethylene diamine having a molecular weight from about 7,500 to about 27,000 wherein at least 40 weight percent of said adduct is poly(oxyethylene), has been found to be particularly advantageous for use in conditioning contact lenses when used in amounts from about 0.01 to about 15 weight percent. Such surfactants are available from BASF Wyandotte Corp., Wyandotte, Mich., under the registered trademark “Tetronic”. The poloxamers are an analogous series of surfactants and are polyoxyethylene, polyoxypropylene block polymers available from BASF Wyandotte Corp., Parsippany, N.J. 07054 under the trademark “Pluronic”.

The HLB of a surfactant is known to be a factor in determining the emulsification characteristics of a nonionic surfactant. In general, surfactants with lower HLB values are more lipophilic, while surfactants with higher HLB values are more hydrophilic. The HLB values of various poloxamines and poloxamers are provided by BASE Wyandotte Corp., Wyandotte, Mich. Preferably, the HLB of the surfactant in the present invention is at least 18, more preferably 18 to 32, based on values reported by BASF.

Additional compatible surfactants that are known to be useful in contact wetting or rewetting solutions can be used in the solutions of this invention. The surfactant should be soluble in the lens care solution and non-irritating to eye tissues. Satisfactory non-ionic surfactants include polyethylene glycol esters of fatty acids, e.g. coconut, polysorbate, polyoxyethylene or polyoxypropylene ethers of higher alkanes (C₁₂-C₁₈ ). Examples of the preferred class include polysorbate 20 (available from ICI Americas Inc., Wilmington, Del. 19897 under the trademark Tween® 20), polyoxyethylene (23) lauryl ether (Brij® 35), polyoxyethylene (40) stearate (Myrj® 52), polyoxye thylene (25) propylene glycol stearate (Atlas® G 2612). Brij® 35, Myrj® 52 and Atlas® G 2612 are trademarks of, and are commercially available from, ICI Americas Inc., Wilmington, Del. 19897.

Various other surfactants suitable for in the invention can be readily ascertained, in view of the foregoing description, from McCutcheon's Detergents and Emulsifiers, North American Edition, McCutcheon Division, MC Publishing Co., Glen Rock, N.J. 07452 and the CTFA International Cosmetic Ingredient Handbook, Published by The Cosmetic, Toiletry, and Fragrance Association, Washington, D.C. however, the preferred surfactants are commercially available surfactants sold under the trademark Cremaphor RH40® by BASF which are polyoxyethoxylated castor oils.

### EXAMPLES

The following examples illustrate the inventor but do not fully delineate the scope of the invention intended by the inventor to be claimed herein. They are intended to illustrate how the invention might be practiced in certain particulars, but are not meant to be interpreted by those of skill in this art restrictively.

### Example 1

Histidine-Peroxide

Formulations were prepared by dissolving L-histidine in water. The pH of the solutions were adjusted to 7.3 with 1N hydrochloric acid. Hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | | | Hydrogen | Dequest |
|---|---|---|---|---|
| Reduction | Preservative | Buffer | Peroxide | 2010 |
| 1.25 | PHMB 0.0001% | L-histidine 0.2% | none | 0.006% |
| 1.85 | PHMB 0.0001% | L-histdine 0.2% | 0.006% | 0.006% |

The results demonstrate the improved antifungal efficacy of the histidine-hydrogen peroxide combination against C. albicans.

### Example 2

Histidine-Peroxide

Formulations were prepared by dissolving L-histidine in water. The pH of the solutions were adjusted to 7.3 with 1N hydrochloric acid. Sodium chloride, Hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | Preser- | | Sodium | Hydrogen | Dequest |
|---|---|---|---|---|---|
| Reduction | vative | Buffer | Chloride | Peroxide | 2010 |
| 0.50 | PHMB | L-histdine | 0.4% | none | 0.006% |
| | 0.0001% | 0.2% | | | |
| 1.08 | PHMB | L-histdine | 0.4% | 0.006% | 0.006% |
| | 0.0001% | 0.2% | | | |

The results demonstrate the improved antifungal efficacy of the histidine-hydrogen peroxide combination against C. albicans.

### Example 3

Histidine-Peroxide

Formulations were prepared by dissolving L-histidine in water. The pH of the solutions were adjusted to 7.3 with 1N hydrochloric acid. Glycerin, hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | Preser- | | | Hydrogen | Dequest |
|---|---|---|---|---|---|
| Reduction | vative | Buffer | Glycerin | Peroxide | 2010 |
| 1.60 | PHMB | L-Histidine | none | none | none |
| | 0.0001% | 0.2% | | | |
| 2.38 | PHMB | L-Histidine | none | 0.006% | none |
| | 0.0001% | 0.2% | | | |
| 1.27 | PHMB | L-Histidine | none | none | 0.006% |
| | 0.0001% | 0.2% | | | |
| 2.25 | PHMB | L-Histidine | none | 0.006% | 0.006% |
| | 0.0001% | 0.2% | | | |
| 1.08 | PHMB | L-Histidine | none | none | 0.003% |
| | 0.0001% | 0.2% | | | |
| 2.04 | PHMB | L-Histidine | none | 0.006% | 0.003% |
| | 0.0001% | 0.2% | | | |
| 1.57 | PHMB | L-Histidine | 0.50% | none | none |
| | 0.0001% | 0.2% | | | |
| 2.15 | PHMB | L-Histidine | 0.50% | 0.006% | none |
| | 0.0001% | 0.2% | | | |
| 1.25 | PHMB | L-Histidine | 0.50% | none | 0.006% |
| | 0.0001% | 0.2% | | | |
| 2.04 | PHMB | L-Histidine | 0.50% | 0.006% | 0.006% |
| | 0.0001% | 0.2% | | | |
| 1.08 | PHMB | L-Histidine | 0.50% | none | 0.003% |
| | 0.0001% | 0.2% | | | |
| 1.93 | PHMB | L-Histidine | 0.50% | 0.006% | 0.003% |
| | 0.0001% | 0.2% | | | |

The results demonstrate the improved antifungal against C. albicans in each paired formulation, when 0.006% hydrogen peroxide is added. The data demonstrates that the increased activity is independent of the presence of Dequest 2010.

### Example 4

Histidine-Peroxide

Formulations were prepared by dissolving L-histidine in water. The pH of the solutions were adjusted to 7.3 with 1N hydrochloric acid. Hydrogen peroxide, Dequest 2010 and polyhexamethylenebiguanide HCl (PHhMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | | | Hydrogen | Dequest |
|---|---|---|---|---|
| Reduction | Preservative | Buffer | Peroxide | 2010 |
| 2.01 | PHMB 0.0001% | Histidine 0.2% | none | none |
| 2.42 | PHMB 0.0001% | Histidine 0.2% | 0.006% | 0.003% |
| 0.73 | Marketed | | | |
| | Product 1 | | | |
| 1.95 | Marketed | | | |
| | Product 2 | | | |

* marketed product 1 having the general composition: A sterile isotonic aqueous solution containing sodium chloride, polyoxyethylene polyoxypropylene block copolymer, sodium phosphate dibasic, sodium phosphate monobasic, and preserved with edetate disodium dihydrate 0.025% and polyhexanide 0.0001%.

** marketed product 2 having the general composition: A sterile, isotonic solution that contains HYDRANATE (hydroxyalkylphosphonate), boric acid, edetate disodium, poloxamine, sodium borate and sodium chloride; preserved with DYMED (polyaminopropyl biquanide) 0.0001%.

The results demonstrate the improved antifungal efficacy of the histidine-hydrogen peroxide combination. The effectiveness was superior to that found in either commercially marketed products.

### Example 5

Histidine-Peroxide

Formulations were prepared by dissolving L-histidine in water, The pH of the solutions were adjusted to 7.3 with 1N hydrochloric acid. Cremophor RH40, hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| Log | Preserva- | | | Hydrogen | Dequest |
|---|---|---|---|---|---|
| Reduction | tive | Buffer | Additive | Peroxide | 2010 |
| 2.51 | PHMB | L-Histidine | Cremophor | none | none |
| | 0.0001% | 0.2% | RH 40 | | |
| 3.27 | PHMB | L-Histidine | Cremophor | 0.006% | 0.003% |
| | 0.0001% | 0.2% | RH 40 | | |

The results demonstrate the improved antifungal efficacy of the histidine-hydrogen peroxide combination against C. albicans.

### Example 6

Histidine-Peroxide

Formulations were prepared by dissolving L-histidine in water. The pH of the solutions were adjusted to 7.3 with 1N hydrochloric acid. The tonicity agent, hydrogen peroxide, Dequest 2010 and polyhexamethylenebiguanide HCl (PHMB) were added to these solutions. The formulations were diluted to volume with water. Each of these solutions were tested for their activity against C. albicans (ATCC 10231) following a two hour exposure. The activity is expressed as a log reduction from the initial inoculum. The compositions, concentrations and activity of each of the solutions are summarized in the following table.

| | | | Tonicity | | | |
|---|---|---|---|---|---|---|
| Log Reduction | Preservative | Buffer | Agent | Wetting Agent | Hydrogen Peroxide | Dequest 2010 |
| 2.42 | PHMB 0.0001% | L-Histidine 0.2% | none | Cremophor RH 40 | | |
| 3.34 | PHMB 0.0001% | L-Histidine 0.2% | none | Cremophor RH 40 | 0.006% | 0.003% |
| 2.19 | PHMB 0.0001% | L-Histidine 0.2% | glycerin 3% | Cremophor RH 40 | | |
| 2.94 | PHMB 0.0001% | L-Histidine 0.2% | glycerin 3% | Cremophor RH 40 | 0.006% | 0.003% |
| 2.19 | PHMB 0.0001% | L-Histidine 0.2% | propylene glycol 3% | Cremophor RH 40 | | |
| 2.95 | PHMB 0.0001% | L-Histidine 0.2% | propylene glycol 3% | Cremophor RH 40 | 0.006% | 0.003% |
| 3.36 | PHMB 0.0001% | L-Histidine 0.2% | sorbitol 5% | Cremophor RH 40 | | |
| 3.92 | PHMB 0.0001% | L-Histidine 0.2% | sorbitol 5% | Cremophor RH 40 | 0.006% | 0.003% |

| | |
|---|---|
| 0.68 | Marketed Product 1 |
| 2.99 | Marketed Product 2 |
| 2.98 | Marketed Product 3 (Opti-Free Express) |

* marketed product 1 having the general composition: A sterile isotonic aqueous solution containing sodium chloride, polyoxyethylene polyoxypropylene block copolymer, sodium phosphate dibasic, sodium phosphate monobasic, and preserved with edetate disodium dihydrate 0.025% and polyhexanide 0.0001%.

** marketed product 2 having the general composition: A sterile, isotonic solution that contains HYDRANATE (hydroxyalkylphosphonate), boric acid, edetate disodium, poloxamine, sodium borate and sodium chloride; preserved with DYMED (polyaminopropyl biquanide) 0.0001%.

The data shows that the addition of 0.006% hydrogen peroxide to histidine provides increased antifungal ctivity against C. albicans. Consistent results were found in the presence of Cremophor RH40 with glycerin, propylene glycol, and soribitol. All formulations with dilute hydrogen peroxide added to hisitidine were equal to or superior to marketed products.

What is claimed is:

1. An ophthalmic solution comprising:
0.00001 to about 10.0 percent by weight L-histidine, and
0.0001 to 3.0 percent by weight hydrogen peroxide.

2. The ophthalmic solution of claim 1, further comprising 0.1 to 500 parts per million of a preservative.

3. A method for supplying a rinsing solution to an eye comprising the step of:
Contacting an eye with a solution comprising:
0.00001 to about 10.0 percent by weight L-histidine, and
0.0001 to 3.0 percent by weight hydrogen peroxide.

4. The method of claim 3, wherein said solution further comprises 0.1 to 500 parts per million of a preservative.

5. A contact lens solution comprising:
0.00001 to about 10.0 percent by weight histidine, and
0.0001 to 3.0 percent by weight hydrogen peroxide.

6. The contact lens solution of claim 5, further comprising 0.1 to 500 parts per million of a preservative.
